(19) United States
(12) Patent Application Publication
(10) Pub. No.: US 2004/0259221 A1
(43) Pub. Date: Dec. 23, 2004

(54) Separation of cytosolic and nuclear proteins

(75) Inventor: Ming Zhao, North Potomac, MD (US)

Correspondence Address: LARGE SCALE BIOLOGY CORPORATION, 3333 VACA VALLEY PARKWAY, SUITE 1000, VACAVILLE, CA, 95688, US

(73) Assignee: LARGE SCALE BIOLOGY CORPORATION, Vacaville, CA (US)

(21) Appl. No.: 10/850,734

(22) Filed: May 21, 2004

Related U.S. Application Data
(60) Provisional application No. 60/472,376, filed on May 21, 2003.

(51) Int. Cl. C12N 9/10; C12N 9/22

(52) U.S. Cl. 435/199; 530/358

(57) ABSTRACT

Methods and compositions for subcellular fractionation of biological samples before proteomic analysis are given. Of particular interest is the separation of DNA binding nuclear proteins.

### CROSS-REFERENCE TO RELATED APPLICATIONS

This application is based on, and claims the benefit of, U.S. Provisional Application No. 60/472,376, filed May 21, 2003, and entitled, PREPARATION OF CYTOSOLIC AND NUCLEAR PROTEINS and which is incorporated herein by reference.

This invention was made with United States Government Support under Contract number NO1-ES-25494 awarded Jul. 19, 2002 by the National Institute of Environmental Health Sciences. The United States Government has certain rights to this invention.

### FIELD OF THE INVENTION

The present invention relates the separation of proteins from a subcellular fraction. The present invention also relates to the isolation and quantification of many proteins simultaneously within a subcellular fraction.

### BACKGROUND OF THE INVENTION

In the study of proteins within a cellular sample, the location within the cell and concentrations at different locations is of importance to the functioning of the cell. DNA binding proteins are of particular interest as they typically represent the factors responsible for gene regulation and control of the entire cell's metabolism. Other proteins within a cell nucleus have other functions relating to gene expression, DNA replication, repair, and packaging and metabolic signaling to affect gene expression. Because of their critical functions, these proteins are sometimes targets for carcinogens, toxins and potential drugs and the measurement of such proteins may provide an indication of such chemical-protein interactions.

Many of these proteins are not found elsewhere in the cell. The absolute amount of each DNA binding regulatory proteins need not be large because the number of DNA molecule(s) and copies of a gene are generally very few. Other nuclear proteins also have a relatively low abundance compared to general cytoplasmic cellular proteins.

Likewise, certain proteins such as those involved in intracellular signaling are primarily found in the cytoplasm. Membrane proteins found in the cytoplasmic membrane are frequently different, qualitatively or quantitatively, from those found in the nuclear membrane.

Because of this relatively low abundance in whole cells, it may be difficult to separate and isolate sizable amounts of such proteins and quantifying them in the presence of a vast excess of other cellular proteins presents other difficulties. These problems are particularly acute when one lacks a sensitive specific assay for a protein or group of proteins such as a biological assay.

To study unknown nuclear proteins, techniques have been developed to first separate the cell nuclei from the remainder of the cell. Generally, such techniques involve variations on cell lysis and centrifugation through a density gradient, (e.g. WO 01/90719) or by various liquid extraction methods. Deryckere and Gannon developed a mini-preparation method for extraction of DNA-binding proteins from animal tissues (BioTechniques 16(3), 405, 1994) based on cell lysis, centrifugation to pellet nuclei, extraction of nuclear proteins from the pellet.

Other types of proteins have been extracted such as deLarminat et al, Journal of Steroid Biochemistry 16(6):811-6 (1982).

For complete proteomic analysis of a cellular sample, one wishes to detect and quantify many proteins from all portions of the cells. In order to detect lower abundance proteins, many have resorted to sample fractionation to reduce the complexity of the sample (e.g. U.S. Pat. No. 6,391,650) and/or to remove abundant proteins (e.g. WO 02/55654).

High throughput analysis of proteomes by 2-dimensional gel electrophoresis analysis combined with mass spectrometry protein identification has become an important tool in the understanding of mechanisms and functions of biological processes, as well as disease processing and development in this post genomic era. Further, the quality of protein identification by mass spectrometry or other analysis is greatly affected by sample preparation methods.

### SUMMARY OF THE INVENTION

The present invention is a method for sub-cellular fractionation for the preparation of high quality sub-cellular fractions quickly, economically, and reproducibly. Proteomic research requires high quality samples for accurate protein identification and quantification. To this end, the present invention combines a cytosolic-nuclear protein preparation method, which is simple, fast, economic, reproducible, and suitable for high-through-put proteomic research applications.

The object of the present invention is to prepare samples with a quality fractionation to enable detection and measurement of low abundance proteins.

It is a further object of the present invention to analyze nuclear proteins, particularly DNA binding proteins.

It is another object of the present invention to measure multiple proteins simultaneously without loss of accurate quantitative measurements of one or more of the multiple proteins.

It is yet another object of the present invention to reduce handling and increase throughput to perform proteomic analysis of many samples in a practical manner.

It is still another object of the present invention to determine the amounts of different proteins in different subcellular fractions without significant loss of proteins in another subcellular fraction.

It is another further object of the present invention to utilize a volatile buffer, such as ammonium bicarbonate, to avoid the need for desalting.

It is yet another further object of the present invention to perform the fractionation procedure quickly and in a manner which minimizes protein degradation or alteration by proteases, phosphatases and other enzymes and which minimizes handling where very low abundance proteins may be lost due to dilution, adsorption, etc. Of particular concern is a traditional dialysis step to remove salts. This step requires time and allows some protein degradation as well as adsorption of some proteins to the membrane. The result is a degrading of the sample with less accurate measurement of proteins present and their concentrations.

It is a still another object of the present invention to prepare and package at least some of the reagents needed for performing the fractionation into a kit. The kit may also contain vessels and/or other apparatus as well as instructions.

The present method involves lysing cellular membranes in a biological sample while leaving the nuclei intact followed by separation of cytosolic proteins from nuclei. The nuclei, relatively free of cytosolic proteins, are then extracted to solubilize nuclear proteins. Then the resulting fractions may be added to an appropriate electrophoresis buffer and then subjected to 2-D gel electrophoresis such as in U.S. Pat. No. 5,993,627. Alternatively, the fractions may be analyzed by other conventional protein separation and analysis techniques, such as, chromatography alone or coupled with other systems such as mass spectrometry.

### DESCRIPTION OF THE PREFERRED EMBODIMENTS

The term “isolated”, when referring to a protein, means a chemical composition that is essentially free of other cellular components, particularly most other proteins. The term “purified” refers to a state where the relative concentration of a protein is significantly higher than a composition where the protein is not purified. Purity and homogeneity are typically determined using analytical techniques such as polyacrylamide gel electrophoresis or high performance liquid chromatography. Generally, a purified or isolated protein will comprise more than 80% of all macromolecular species present in the preparation. Preferably, the protein is purified to greater than 90% of all macromolecular species present. More preferably, the protein is purified to greater than 95% and most preferably, the protein is purified to essential homogeneity, or wherein other macromolecular species are not significantly detected by conventional techniques. Some proteins may have different forms of the same protein, typically due to variants or post translational modifications. Depending on the resulting data, these may constitute the “same” protein or a different protein.

The term “protein” is intended to also encompass derivitized molecules, such as glycoproteins and lipoproteins, as well as lower molecular weight polypeptides. The term “proteome” is a large number of proteins from a sample or fraction thereof, preferably all detectable by a particular technique or combination of techniques. The proteome will generally have at least 100, preferably over 1000 detectable proteins from a sample when separated by various techniques. In the present invention, the proteome is generated by two-dimensional gel electrophoresis. While this is the generally accepted technique for determining the proteome, other techniques may be acceptable for the present invention if they generate large numbers of quantitatively detectable proteins.

The “sample” in the present invention contains cells of a biological origin. While any eukaryotic cell-containing sample may be used, the present invention is particularly preferred for plant and animal (including human) cells. Fluid samples containing liquids other than cells are included provided that at least some nucleated cells are present, e.g. blood, urine, etc.

The general goal of the present invention is to determine the differential amounts of proteins found in different subcellular locations, which cannot readily be determined by using whole cells. For example a protein may be phosphorylated to a different percentage in one subcellular particle and phosphorylated to a different percentage in another subcellular particle or in the whole cell. Without initial subcellular fractionation, it would be quite difficult to discover such a fact.

Additionally, many protein separation and detection techniques are limited in their sensitivity. Separation methods removing a sizable quantity of unwanted protein serves to increase the relative concentration of the proteins one wishes to measure and thereby increase sensitivity. The same steps may also remove interfering or contaminating materials.

The purpose of the present invention is to generate subcellular fractions to enhance the sensitivity of proteomic analysis techniques. Particularly preferred are 2-dimensional electrophoresis and liquid chromatography combined with mass spectrometry. Other protein separation methods may also be used such as single or multidimensional liquid chromatography, affinity chromatography, gradient surfaces or different affinity binding surfaces, etc. Sequencing, peptide mass determination, binding assays or biological assays are representative of the many ways for identifying the proteins separated. Quantification may be enhanced by the addition of internal standards or compared to controls where the control proteins and test sample proteins are differentially labeled before or after separation. Isotope coded affinity tag labeling is an example of a known quantification technique.

The basic steps involve gentle homogenization of the sample to lyse the cellular membranes but not the nuclei. The nuclei are separated from the cytoplasmic components and then the nuclear proteins are extracted from the nuclei by salt extraction. Other organelles and subcellular structures, such as chloroplasts, mitochondria, glogi, membranes, proteosomes, lysosomes, various complexes etc., can be separated and their proteins extracted using essentially the same technique optimized for each organelle.

The salt being used is preferably volatile and is removed by volatilizing without the need for dialysis. Volatilizing may be performed by lyophilization, gentle heating or vacuum. The salt may be volatilized by being sublimation or by evaporation. Salts, not normally volatile, but which can be made volatile by pH change, chemical reaction, degradation, etc. are also considered to be volatile salts for the purposes of this invention. Removal by dialysis is considered to be any separation step where liquid containing the salt can travel but the proteins cannot. Examples include across a dialysis membrane, across an ultrafilter, into a microporous gel or particle, etc.

The salt is any material that alters the osmotic strength of the solution sufficiently to extract the proteins. Chemicals not usually classified as “salts” may also be used instead of or in conjunction with salts to effect the same change in osmotic strength sufficient to extract the proteins. Examples include certain polymers and organic solvents. In an embodiment of the present invention, these other compositions may be removed by non-dialysis means such as volatilizing, precipitating or reacting with another material to effectively remove them and reduce the osmotic strength of the solution. These situations are considered within the present invention and meaning of removing the salt by volatilizing even if a co-acting salt is not removed.

The concentrations of each reagent in the buffers and solutions are approximate and some buffers, salts, osmotic agents, stabilizers, degradation inhibitors (e.g. inhibitors of protease, lipase, phosphatase, etc.) may be substituted for by others. Some solution components may be present in concentrations up to and more than 10 fold more or less than the amount given provided that they perform the same function. Additionally, stabilizers, cofactors and ions for certain proteins in the samples may be added. Additional components include those which will reduce interference from other components in the sample such as adding Dnase and/or Rnase to degrade nucleic acids, detergents to solubilize lipids, etc. Common abundant proteins, contaminants, and/or unwanted sample proteins may be removed by specific (e.g. WO 02/055654) or non-specific binding agents (e.g. Cibachrome Blue).

When attempting to determine proteins bound to other structures, such as DNA or membranes, the entire structure may first be isolated followed by degradation or separation from the structure. Such separation from a subcellular particle may use any of the conventional techniques known per se.

While the specification and examples are directed toward recovery of proteins, nucleic acids and other cellular components may also be recovered and analyzed.

### EXAMPLE 1

### Homogenization of Rat Liver Samples

Fresh rat liver was cut into small pieces and briefly rinsed with ice-cold phosphate buffered saline with 1 mM PMSF to remove blood. The tissue was stored at −70° C. and/or kept on dry ice until use. Alternatively, the tissue was immediately washed in the buffer as indicated above, rapidly frozen in liquid nitrogen, crushed into a granular substance using a tissue grinder or mortar and frozen at −70° C.

Approximately 2.5 g of tissue was weighed, ice-cold Homogenization Buffer of 10 mM HEPES pH 7.9, 0.25 M sucrose, 2 mM MgCl₂, 1× EDTA-free protease inhibitor cocktail (Roche), 1 mM β-Glycerophosphate; was added to cover the tissue material before it was sliced into smaller pieces with a surgical bladder on three layers of ice-cold aluminum wrap. The tissue was transferred into a 50 ml Falcon centrifuge tube sitting on ice pre-filled with 12.5 ml Homogenization Buffer. If tissue crushed in liquid nitrogen is available, the frozen material was weighed and directly transferred into a 50 ml tube sitting on ice pre-filled with 12.5 ml Homogenization Buffer.

Sliced or crushed tissue was homogenized with 15 strokes on ice in a Dounce homogenizer. The suspension was set aside on ice for 15 min. The suspension was then transferred into a 15 ml Falcon centrifuge tube and centrifuged at 1,000×g for 10 min. The supernatant (cytosolic fraction, approximately 12 ml) and the pellet (nuclear fraction, approximately 3 ml) were collected separately.

### EXAMPLE 2

### Isolation and Preparation of Cytosolic Fraction for 2-D Gel Electrophoresis

The supernatant from the 1,000×g centrifugation was transferred into ice-cold Beckman polycarbonate ultracentrifuge tubes (16×76 mm). The tubes were placed in 70.1 Ti rotor chilled at 4° C. and, in an Optima L-70K centrifuge, the homogenate was centrifuged at 100,000×g (45,000 rpm) for 1 hour.

Using disposable transfer pipettes, the clear supernatant was collected while avoiding disturbing a floating layer of lipids. The supernatant was transferred into 1.5 ml microtubes and centrifuged again at 10,000×g for 30 min to remove residual lipid adhering to the walls of the microtubes. Clear supernatant was collected and aliquoted for storage. A 100μl sample was retained for measurement of the protein concentration and SDS-PAGE loading 20μg of the protein mixture.

In preparation of a sample for two-dimensional electrophoresis (2-DE), the volume of cytosolic supernatant needed to generate about 300 mg/ml cytosolic protein samples. The more diluted supernatant obtained after high-speed centrifugation is concentrated by ultrafiltration in an Ultrafree-4/5K unit. Twice the volume of 2-DE CHAPS solubilization buffer as described in U.S. Pat. No. 5,993,627 was added and the concentrate was recovered from the Ultrafree tube. The protein concentration was adjusted to 40 mg/ml with diluted solubilization buffer, which contains 67% 2-DE CHAPS buffer, and 33% water and stored at −70° C. for 2-DE gel analysis.

### EXAMPLE 3

### Purification of Nuclear Fraction from Membranes and Cytoplasmic Contaminants

The pellet from the 1000×g centrifugation step (about 3.0 ml in the beginning) was re-suspended with Wash Buffer, 10 mM HEPES, 2 mM MgCl₂, 0.6% NP-40, pH7.9, 5 mM NaCl, 0.5× protease inhibitor cocktail, 0.5 mM β-Glycerophosphate and brought up to 10 ml with same buffer. The suspension was set aside on ice for 10 min with occasional shaking (approximately once every two minutes). The suspension is centrifuged at 1000×g for 7 min. The pellet is collected and the supernatant discarded. This suspension/centrifugation procedure is repeated four times.

The pellet from the fourth centrifugation was suspended again in 4 ml Wash Buffer and set aside on ice for 3-4 minutes. The top layer of the suspension was transferred to another 15 ml tube. The bottom layer of the suspension, which contains the majority of the pelleted material, particularly clumps of connective tissue, was re-suspended by adding 3 ml of Wash Buffer and inverted twice. After about 2-3 minutes, the top portion was collected. The bottom layer was re-suspended by adding 3 ml of Wash Buffer and, again, set aside on ice for about 1-2 minutes. The top layer was carefully collected, while avoiding disturbing the bottom collective tissues.

The collected top layers of the above-described procedures contain the nuclei. They were combined and centrifuged at 1000×g for 7 min. The supernatants were removed and the pellets centrifuged for an additional 4 min at 2500×g. 1.5 ml ice-cold Extraction Buffer, 440 mM ammonium bicarbonate, 0.2 mM MgCl₂, 1 mM β-mercaptoethanol, 0.3× EDTA-free protease inhibitor cocktail, 0.1 mM β-Glycerophosphate, 25 mM sucrose) was added to the pellet and re-suspended immediately by pipetting. The tube was set aside on ice for 30 min, while occasionally flicking the tube.

The extraction suspension was transferred to 1.5 ml microtubes and centrifuged at 11,000×g for 30 min. The supernatant was collected and filtrated through centrifugal filtration device with membranes of 0.45 μm pore size (Ultrafree CL). A small aliquot was taken to measure protein concentration. The filtrate was lyophilized overnight. 2-DE CHAPS buffer was added to solublize the lyophilized powder. The concentration of the solublized proteins was adjusted to 20 mg/ml. The sample was stored at −70° C. and thawed for 2-DE gel analysis by the techniques described in U.S. Pat. No. 5,993,627.

### EXAMPLE 4

### Nuclear Protein Extraction Kit

A package containing four vessels is prepared for the purpose of performing the extraction method of the present invention. The kit contains:

1. A bottle containing 100 ml of phosphate-buffered saline (PBS) with 1 mM PMSF.

2. A bottle containing 150 ml of Homogenization Buffer with 10 mM HEPES pH 7.9, 0.25 M sucrose, 2 mM MgCl₂, 1× EDTA-free protease inhibitor cocktail (Roche) and 1 mM β-Glycerophosphate.

3. A bottle containing 550 ml of Wash Buffer with 10 mM HEPES, 2 mM MgCl₂, 0.6% NP-40, pH 7.9, 5 mM NaCl, 0.5 × protease inhibitor cocktail and 0.5 mM β-Glycerophosphate.

4. A bottle containing 20 ml of Extraction Buffer with 440 mM ammonium bicarbonate, 0.2 mM MgCl₂, 1 mM β-mercaptoethanol, 0.3× EDTA-free protease inhibitor cocktail, 0.1 mM β-Glycerophosphate and 25 mM sucrose.

5. Optionally, a bottle containing protease inhibitor cocktail when the protease inhibitor cocktail is omitted from bottles 2-4 above.

The kit may also include instructions, centrifuge tubes, ultracentrifuge tubes and Eppendorf microfage tubes. This package is designed to perform 10 extractions.

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

All patents and references cited herein are explicitly incorporated by reference in their entirety.

What is claimed is:

1. A method for recovering and separating nuclear proteins from a cellular sample comprising;
separating cell nuclei from cytoplasmic proteins,
recovering the cell nuclei,
extracting nuclear proteins from the cell nuclei, and
separating extracted nuclear proteins,
wherein a dialysis step is not present between said extracting and said separating extracted nuclear proteins.

2. The method of claim 1 wherein said separating nuclei from cytoplasmic proteins is performed in a buffered solution containing protease inhibitors.

3. The method of claim 1 wherein said extracting nuclear proteins is performed in a buffered solution containing protease inhibitors.

4. The method of claim 3 wherein the buffer in the buffered solution is volatile.

5. The method of claim 4 wherein the buffer is ammonium bicarbonate.

6. The method of claim 4 further comprising removing the volatile buffer before separating extracted nuclear proteins.

7. The method of claim 1 wherein the nuclear proteins are DNA binding proteins.

8. The method of claim 1 wherein the separating extracted nuclear proteins is performed by electrophoresis.

9. The method of claim 1 wherein the separating extracted nuclear proteins is performed by chromatography.

10. The method of claim 1 further comprising identifying individual separated extracted nuclear proteins.

11. The method of claim 10 wherein the quantity of the separated extracted nuclear proteins is determined.

12. The method of claim 1 further comprising recovering the cytoplasmic proteins and separating the cytoplasmic proteins.

13. The method of claim 12 wherein the separating the cytoplasmic proteins is performed by electrophoresis.

14. The method of claim 12 wherein the separating the cytoplasmic proteins is performed by chromatography.

15. A kit for obtaining proteins from a biological sample containing eukaryotic cells comprising;
a vessel containing a protein extraction buffer wherein the buffering salt is volatile, and
instructions for use of the extraction buffer to extract proteins from a subcellular fraction and to remove the buffering salt by volatilizing.

16. The kit of claim 15 further comprising a vessel containing a homogenization buffer for lysing and separating subcellular particles from cytoplasm.

17. The kit of claim 16 further comprising a vessel containing wash buffer for washing subcellular particles.

18. The kit of claim 15 further comprising protease inhibitors in the same vessel or in a separate vessel.

19. The kit of claim 16 wherein the buffering salt is ammonium bicarbonate.

20. The kit of claim 15 wherein the subcellular particles are nuclei and the proteins being extracted are nuclear proteins.

21. A composition comprising a mixture of nuclear proteins extracted by the process of claim 1 before the nuclear proteins are separated from each other.

22. The composition of claim 21 wherein the nuclear proteins include DNA binding proteins.

23. A combination of two different 2-dimensional electrophoresis gels, one containing separated cytoplasmic proteins and another containing separated nuclear proteins, wherein the proteins in each of the two gels are from the same individual sample.

24. A method for extracting DNA binding proteins from a complex of DNA and DNA binding proteins comprising;
contacting said the complex of DNA and DNA binding proteins with a buffer containing a volatile salt,
separating the DNA binding proteins from the DNA, and
removing the volatile salt by volatilizing, wherein a dialysis step is not present to remove a salt.

25. The method of claim 24 further comprising separating and identifying the DNA binding proteins.

26. The method of claim 25 further comprising quantitatively measuring the amount of the DNA binding proteins.
